(19) United States
(12) Patent Application Publication
(10) Pub. No.: US 2014/0271783 A1
(43) Pub. Date: Sep. 18, 2014

(54) PREFABRICATED PHARMACEUTICAL DOSAGE FORMS FROM FUNCTIONAL POLYMER FILMS

(71) Applicant: PURDUE RESEARCH FOUNDATION, West Lafayette, IN (US)

(72) Inventors: Rodolfo Pinal, West Lafayette, IN (US); Bo Zhou, West Lafayette, IN (US); Andrew Otte, West Lafayette, IN (US)

(21) Appl. No.: 14/350,749

(22) PCT Filed: Oct. 15, 2012
(86) PCT No.: PCT/US2012/060203

§ 371 Date: Apr. 9, 2014

Related U.S. Application Data
(60) Provisional application No. 61/547,450, filed on Oct. 14, 2011.

(51) Int. Cl. A61K 9/70; A61K 9/00; A61K 31/343

(52) U.S. Cl. CPC A61K 9/7007; A61K 31/343; A61K 9/0056
USPC 424/441; 424/472; 424/457; 424/489; 514/462; 156/228

(57) ABSTRACT

A combination of sheets can be combined into an ingestible unit. The individual sheets can be prepared to have one or more functionalities, such as providing a biologically active agent, disintegrating and opening the unit, controlling release of an agent, facilitating absorption from the GI tract, as well as many others. The individual sheets can be selectively identified for combining into a multifunctional ingestible unit with a random or predetermined arrangement or stacking pattern. The individual sheets can be loose in a capsule or laminated together into a stacked layered structure. The combination of sheets can be pressed, laminated, tableted, or otherwise prepared into an ingestible unit. The ingestible unit can be predetermined to be useful for administering a drug, drug combination, multi-drug regimen as well as tailored to subject-specific multi-drug therapeutic regimens. The sheets can be loaded with any type of agent ranging from drugs to anti-counterfeit agents.

### CROSS REFERENCE TO RELATED APPLICATIONS

This patent application claims benefit of U.S. Provisional Patent Application Ser. No. 61/547,450 filed Oct. 14, 2011, which is incorporated herein by specific reference in its entirety.

### GOVERNMENT RIGHTS

This invention was made with government support under EEC-0540855 and RU4-29365 10221 awarded by the National Science Foundation. The government has certain rights in the invention.

### BACKGROUND

The vast majority of pharmaceutical and nutraceutical products are administered by the oral route. The oral route of administration is convenient from the patient's point of view since administration is as simple as swallowing a solid oral dosage form, such as a tablet or capsule. Tablets and capsules are a commonly-used dosage form used to deliver drugs; however, many different types of dosage formats exist. Traditional tablets are made by compacting powder blends made from the API (active pharmaceutical ingredient) and other excipients, or pharmacologically inactive ingredients, such as fillers, agglutinants, lubricants, glidants, and disintegrants. Some ingredients, such as agglutinants, lubricants, and glidants are present simply for purposes of processing and manufacturing the tablet. This means that their presence is not the result of any consideration pertaining directly to the desired pharmaceutical performance of the final product. Capsules often have a mixture of powders, where powder handling and quality control can be difficult in pharmaceutical or nutraceutical manufacturing. While previous oral dosage forms have been suitable, there always remains a need and desire for improvement.

### BRIEF DESCRIPTION OF THE FIGURES

The foregoing and following information as well as other features of this disclosure will become more fully apparent from the following description and appended claims, taken in conjunction with the accompanying drawings.

FIG. 1 illustrates different embodiments of loose functional sheets, laminated functional sheets, and shaped laminated functional sheets.

FIG. 2A illustrates an embodiment of a method of casting a composition into a film, and shaping the film into a defined shape.

FIG. 2B illustrates an embodiment of an extrusion system for manufacturing functional sheets.

FIG. 2C illustrates an embodiment of a molding system for manufacturing functional sheets.

FIG. 3 illustrates an image of an embodiment of a functional sheet including particles suspended in a sheet matrix.

FIG. 4 shows a raman-based chemical imaging picture of a surface of an embodiment of a sheet having particles of griseofulvin (white) suspended in a matrix of HPMC (hydroxypropylmethyl cellulose; dark).

FIG. 5A provides a graph that shows the amount of dissolved griseofulvin from HPMC films and alginate films loaded at 9.3% compared to pure drug films.

FIG. 5B provides a graph that shows the percent of dissolved griseofulvin from HMPC films with agglomerated particles compared to HMCP films without any agglomerated particles from 9.3% loadings.

FIG. 5C provides a graph that shows the percent of dissolved griseofulvin from HMPC and/or SA LVCR films with 9.3% griseofulvin.

FIG. 5D provides a graph that shows the percent of dissolved griseofulvin as a function of film thickness.

FIG. 6 provides a graph that shows accelerated drug release rate of griseofulvin when drug particles are spatially dispersed and immobilized in a polymer film matrix.

FIG. 7 provides a graph that shows that even at high load of drug particles in the film (about 72% w/w), the non-agglomerating effect of the film configuration is observed by the continued increase in dissolution rate with drug load.

FIG. 8A provides a graph that shows the large change in drug release rate obtainable by adjusting the molecular weight of a HPMC polymer.

FIG. 8B provides a graph that shows the effect of using a mixed HPMC/alginate polymer matrix on drug release rate from the obtained film.

FIG. 9A provides a graph that shows an example where particle-containing films made from sodium alginate match the time to saturation of a commercial product.

FIG. 9B provides a graph that shows that keeping the same polymer chemistry (e.g., Methocel® K series) but changing the molecular weight is an effective method for modifying the drug release attributes of the multilayer ingestible units.

FIG. 9C provides a graph that shows an example of the release profile when a layer of disintegrant is included.

FIG. 10 provides a graph that shows the synopsis of the versatility offered by the multilayer ingestible units.

FIG. 11 provides a graph that shows the changes with constant drug load and different molecular weights of a polymer.

FIG. 12 provides a graph showing the same polymer with different drug loads.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention relates to a combination of sheets that are combined into an ingestible unit. The individual sheets can be prepared to have one or more functionalities, such as providing a biologically active agent, disintegrating and opening the unit, controlling release of an agent, facilitating absorption from the GI tract, as well as many others. The individual sheets can be selectively identified for combining into a multifunctional ingestible unit with a random or predetermined arrangement or stacking pattern. The individual sheets can be loose in a capsule or laminated together into a stacked layered structure. The combination of sheets can be pressed, adhered, glued, affixed, laminated, tableted, or otherwise prepared into an ingestible unit. The ingestible unit can be predetermined to be useful for administering a drug, drug combination, multi-drug regimen as well as tailored subject-specific drug release profile and/or multi-drug therapeutic regimens. In one aspect, the layered-sheet configuration can allow for ingestible units to be prepared so as to be devoid of tablet components that are not useful for the therapy, such as by being devoid of agglutinants, lubricants, glidants, and the like used for tablet manufacturing.

In one embodiment, the present invention includes an ingestible unit, which can be a customized multifunctional pharmaceutical or nutraceutical dosage format or placebo. The ingestible unit is capable of being ingested, either orally, vaginally, or rectally. The ingestible unit can be sized so that it can be swallowed without chewing, whether taken dry or administered with a liquid chaser. The ingestible unit can include a plurality of discrete sheets combined into an ingestible unit, wherein each discrete sheet has a composition configured for one or more predetermined functions. For example, different sheets can be predesigned for different functions and combined into a multifunctional ingestible unit, where each sheet can contribute a function based on composition and/or arrangement within the ingestible unit. The sheets can either be laminated together into a laminate structure or packaged loosely together in an ingestible container, such as a capsule. The sheets can be selected to combine one or more predetermined functions such as to: provide a filler; provide a taste-masking agent; provide color to the unit; solubilize a biologically active agent; provide for effervescence; provide an antioxidant; provide an absorption enhancer; provide a transporter inhibitor; provide a transporter inducer; provide a surfactant; provide an emulsifying agent; provide a self-emulsifying system; provide a crystallization inhibitor; provide a supersaturation promoting agent; provide an antimicrobial preservative; provide a pH modifying or buffer agent; provide a catalyst; provide a complexing agent for a biologically active agent; provide a chelating agent, provide a bioadhesive agent; provide particles having a bioactive agent; control rate of bioactive agent release; provide mechanical strength; provide flexibility; provide rigidity; provide an organoleptic agent such as a flavor and/or a scent; provide radiotranslucency; provide radiopaquness; provide a tracking substance; provide a tracking device; provide an identifying substance; provide an identifying device; provide an anti-counterfeiting substance; provide an anti-counterfeiting pattern; provide and anti-counterfeiting device; provide water-dissolvability; provide water-stability; provide for disintegration; provide an osmotic agent; provide immediate release of an agent; provide delayed release of an agent; provide sustained release of an agent; and/or provide for separation of the unit into two or more parts. The individual sheets can have single functions or multiple functions.

In one embodiment, the ingestible unit can include at least one discrete sheet of a first type and at least one discrete sheet of a second type different from the first type, where the different types can have different functions or different characteristics or differences of a characteristic. The first type and second type can have first and second special distributions within the ingestible unit. The first type and second type can be at predetermined locations within the ingestible unit and with respect to each other for customized combined functionalities. In one aspect, the discrete sheets of the first type can include a substance that is devoid in the second type. In one aspect, at least one discrete sheet of a third type that is different from the first type and second type can be included in the ingestible unit. The third type may have a third spatial distribution or be at predetermined locations relative to other sheets, such as those of the first and second types. In one aspect, each of the discrete sheets in the ingestible unit can be substantially identical, such as in a placebo.

In one embodiment, the ingestible unit can have at least one discrete sheet that includes a biologically active agent. The biologically active agent can be any agent that is administered for a function, such as a biological function to improve or otherwise modulate a biological process, such as a biological pathway. However, the agent can be active, such as to emit light, without being biologically active. As such, the biologically active agent can be a traditional pharmaceutical or nutraceutical, and it can be any type of substance for testing or diagnostics. The biologically active agent can be any agent that is administered to a subject in order to elicit a biological response that arises from the biological activity of the agent. The biological response obtained can be a measurable biological response or provide some change that can be analyzed and determined, such as by testing to determine an amount of the biologically active agent to be administered. The biologically active agent can be a toxin or poison or other deleterious substance. Examples can include the biologically active agent being a mineral, vitamin, pharmaceutical, nutraceutical, small molecule, macromolecule, organic molecule, polypeptide, protein, nucleic acid, polynucleotide, derivatives thereof, and combinations thereof. The biologically active agent can be for a human or animal subject. Human and veterinary medicines can be improved with the present invention. Alternatively, each discrete sheet can be devoid of a biologically active agent when the ingestible unit is a placebo.

In one embodiment, the unit having the plurality of sheets can be configured for a use other than administering to a subject. For example, the unit can be configured as an agricultural unit that releases agents into an agricultural environment. In another example, the unit can be pesticidal unit that includes a pesticide, where the unit can release pesticide or be configured to be ingested by a pest. In another example, the unit can be a herbicidal unit that includes a herbicide. In another example, the unit can be a fertilizer unit that includes a fertilizer. These units can be used as is common with these types of compositions. Accordingly, the biologically active agent described herein can be substituted with a different type of functional agent for a different function. Also, the unit can be configured to be used in an environmental setting. As such, the sheets can be configured for agricultural use, and not ingestion. That is, a unit can have one or more sheets can include herbicides, pesticides, and/or fertilizers as agents. The unit can be environmentally dissolvable, such as by being water-dissolvable, or otherwise naturally degradable. Many of the sheets described herein can be used for an agricultural unit.

In one embodiment, the present invention includes a method of making the ingestible units described herein. The method can include obtaining a plurality of the discrete sheets, and combining the discrete sheets into the ingestible unit. The method can include determining the predetermined functions for each discrete sheet. The method can include encasing the discrete sheets in an ingestible container to form the ingestible unit, whether the discrete sheets are loose, random, stacked, arranged, and/or laminated. The individual discrete sheets can be made by any suitable process for preparing thin sheets or films that can be laminated together, such as forming each discrete sheet by one or more of: casting; spin coating; extrusion and calendering; roll-pressing; microfabrication; molding; jet-printing; drop-on-demand; or combinations thereof, or the like. The method can include stacking the plurality of discrete sheets and laminating the plurality of discrete sheets into a laminated body, where optionally the discrete sheets are arranged in a predetermined order.

In one embodiment, the ingestible unit can be prepared to include two or more biologically active agents. A first type of sheet can include a first biologically active agent and a second type of sheet can include a second biologically active agent. The amount and arrangement of the first and second types of sheets can be predetermined. The ingestible unit may include only one biologically active agent. For example, the method can include: determining at least one biologically active agent to be included in the ingestible unit by being included in one or more discrete sheets; determining an amount of the biologically active agent in a discrete sheet; determining a number of discrete sheets having the biologically active agent to combine to obtain the determined dose; and combining the number of discrete sheets having the biologically active agent in the ingestible unit. The biologically active agent can be dispersed within the sheet as discrete molecules or agglomerated or otherwise combined into particles, such as nanoparticles, microparticles, coated particles, nano-capsules or microcapsules where the particles are suspended within the sheet.

In one embodiment, the present invention includes a method of providing an ingestible unit to a subject, where the ingestible unit can be administered for a therapeutic or placebo effect. The method can include providing the ingestible unit as described herein, and administering the ingestible unit to a subject. The biologically active agent can be administered orally, vaginally, or rectally in a therapeutically effective amount to inhibit or treat a disease.

The present invention can include an ingestible unit such as tablets having laminated sheets or capsules having loose sheets made from more than one sheet (e.g., polymer films or inorganic laminates). The sheets, each independently or together, have a specific and predetermined pharmaceutical function in the final ingestible unit format. The present invention can include an ingestible unit that has multiple films, each having a certain functionality, which are put together in a multi-sheet dosage form, such as laminated tablet, caplet, or capsule, and will have a similar look and feel as a traditional tablet or capsule.

FIG. 1 illustrates an example of a method 100 of preparing a laminate body 118 having a plurality of predetermined discrete sheets, such as a taste-masking film 110, disintegrating film 112, biologically active agent film 114, and solubilizing film 116. These films can be preselected so that each film has a specific and predetermined function. The function of each film can be selected independently or based on the other films and film functions to be combined and/or arranged. The individual films can have specific functions that are arranged together to promote or enhance the functionality of the individual films. The combination of films may provide for synergistic effects, such as particularly arranging the sheets, exemplified by the stratification of the taste-masking film 110, disintegrating film 112, biologically active agent film 114, and solubilizing film 116. These films can be combined together into a single laminated body 118. The laminated body 118 may be shaped as a generic shape (e.g., rectangle or square or circle) or any arbitrarily chosen or random or irregular shape. The laminated body 118 can be cut into one or more shaped laminated bodies 120 with one or more specific shapes. As shown, the rectangular laminated body 118 is cut into a plurality of sheets (e.g., six sheets). The combination of sheets can be used in the loose format as shown on the left as individual sheets 110, 112, 114, and 116. The combination of sheets can be used as a generic laminate structure such as the laminated body 118. The combination of sheets can be used as a single shaped laminated body 118 or a plurality of shaped laminated bodies 120. As shown, the laminated body 118 includes a top discrete sheet (e.g., 110), a bottom discrete sheet (e.g., 116), and one or more discrete sheets therebetween (e.g., 112 and 114). The intermediate discrete sheets, as well as the surface discrete sheets may be exposed on the sides of the laminated body so that the layers can be seen. Alternatively, a coating can be applied to cover the sides so that the different laminated layers are not visible.

FIG. 2A shows an embodiment of a process 200 for preparing a thin film sheet 218. As shown, a liquid composition 210 having a carrier 212 and an agent 214, which is cast into a substrate 216, and prepared into a thin film sheet 218. The casting can be into a substrate 216 that is a shaped mold or a flat plate as shown for amorphous casting. The liquid composition 210 can have ingredients that are preselected to provide a thin film sheet 218 that has one or more predetermined functionalities. The casting can include any casting steps for casting a polymeric composition into a thin film. The carrier 212 can be any type of carrier that can be cast into a thin film or other sheet as described herein. The agent 214 can be any type of chemical agent or agent particulate.

It should be noted that the same basic method depicted in FIG. 2A may be used for casting films, whether they have different drug load, different drug particle size, or if they are made from different polymers, or from different grades of a given polymer.

FIG. 2B shows an example of a process 220 for preparing the thin film sheets 240. The process 220 can include introducing a liquid composition 210 into a hopper 222 that feeds into a screw auger mixer 224 in an extruder 226 for extruding the liquid composition 210 into an extrudate 230. The extrudate 230 may optionally be cut or pressed into a sheet of the invention. However, the extrudate 230 can be passed through a sizing plate 232 to form sized extrudate 235 that can be cut into a sheet of the invention. The extrudate 230 is shown to be cooled with a water tank 234 jacketing the sizing plate 232, so that the extrudate 230 can be cooled, if needed, while being dimensioned. The sized extrudate 235 can be processed through one or more rollers 236 so as to calender the extrudate into a thin film 237. The thin film 237 can then be cut with a cutting machine 238 into individual thin film sheets 240, which thin film sheets 240 can be stacked as shown. The thin film sheets 240 can be stored as storage-stable individual thin film sheets 240.

FIG. 2C shows an example of a process 250 for preparing molded thin films 256. A mold array 252 having a plurality of molds 254 can receive a liquid composition 210 in order to form the molded thin films 256 dimensioned and shaped as the mold 254. As such, each mold can be shaped to have a specific cross-sectional width and length and a specific depth in order to have a specific volume and shape. The depth can be thin as a film or thickness dimension described herein. The shape can be rectangular as shown as well as being circular, oval, triangular, square, or any other polygon or any other shape ranging from stars, hearts, or other simple shapes to complex shapes, such as animal, plant, or the like.

The film sheets can be shaped while being formed or shaped after being set. The laminates of the film sheets can also be cut into a specific shape before or after the film sheets are laminated together. The shaping can include cutting, stamping, laser-shaping, or any other method of cutting a thin film or a stack of films.

In one embodiment, the ingestible unit can be a multilayered tablet, which can be prepared to be substantially similar in function to traditional pressed tablets. The multilayered tablet can contain any drug. An example can include the drug being griseofulvin, where the dose is the same as the commercially-available product. The multilayered tablet can be of comparable size and shape as conventional tablet dosage forms (e.g., 250 mg of griseofulvin).

In one example, the thin film sheets can be edible polymer films. Such edible polymer films, which can be for delivery of pharmaceutical products, are commercially available. Widely-known examples of the edible polymer films include the Listerine® PocketPaks strips sold as a dry, portable breath-refreshing product, the Gas-X® peppermint-flavored thin strips containing simethicone as the active pharmaceutical ingredient (API), and the first prescription pharmaceutical film strip, Zuplenz®, containing ondansetron as the API, which was approved by the U.S. FDA in 2010 for the treatment of nausea following chemotherapy for example. These individual thin film sheets are prepared for individual consumption. These individual thin film sheets are not provided in an ingestible unit for consumption of multiple sheets. These thin film sheet products are designed as orally disintegrating films. These thin film sheet products are not designed as gastrointestinal tract (GI tract) delivery vehicles. The active agent is dissolved or molecularly dispersed in a polymer, and may have limited dose, which precludes use of medicaments with higher doses. The instant invention offers numerous benefits that overcome the above limitations of single film sheet products.

The present invention can include a plurality of thin films that together are not orally disintegrating. The thin films can include any type of drug, such as those that can be administered by swallowing and absorption in the GI tract. Thin films that alone may be orally dissolving can be combined with one or more other thin films that together inhibit oral dissolution or disintegration and allow for swallowing intact into the stomach. The ingestible unit may also stay intact into the intestine, where it can dissolve, open, or selectively disintegrate in the large or small intestine. The thin film sheets, alone or in combination, can be used for targeting drug release to the small intestine or to the colon. Furthermore, GI tract disintegrating films allow for delayed, sustained, or controlled release formulations to be prepared into the film sheets of the invention. The multi-sheet ingestible unit can be formed by any film-forming substance (e.g., polymer) that is edible or otherwise ingestible. Since the multi-sheet ingestible unit can be prepared into a unit that is meant to be swallowed as any other tablet, the choice of sheet matrix (e.g., polymer) can be used to control the site (e.g., stomach, small intestine, and colon) and rate (e.g., immediate, delayed, or sustained) of drug release. The drug release characteristics are controlled by the choice of chemistry and grade of the matrix material. For example, a polymer molecular weight, degree of branching or cross-linking may be modulated to control the drug release profile from the individual film sheets as well as the laminated body. The sheets of the invention are not limited to fast-dissolving polymers, and can extend to any inorganic substance that can be pressed into a thin ingestible sheet as well as sheets that do not degrade in the GI tract. The film sheets can be digestion-degradable or digestion-stable. Also, GI tract fluid can act to swell the film sheets so as to allow for modulated drug release compared to unswollen film sheets.

In one embodiment, the drug can be used as fine particles. That is, the drug can be agglomerated alone with an agglomerating agent into particles. The particles can be suspended in the film sheet carrier matrix (e.g., polymer matrix). While some drugs may be in a particle format that is suspended in the carrier matrix, some drugs can be dissolved in the carrier matrix. The particles can be micro- and/or nanoparticles. The particles can be microspheres, liposomes, micelles, or other agglomeration of molecules. FIG. 3 shows an embodiment of a thin film sheet 300 having a carrier matrix 310 containing suspended particles 312, and where the surface 316 includes mounds 314 formed from the particles 312 under the surface 316. FIG. 4 shows a raman-based chemical imaging picture of a surface of a sheet 400 having particles 412 of griseofulvin (white) suspended in a matrix 410 of HPMC (hydroxypropylmethyl cellulose; dark).

The multilayer ingestible units of the instant invention can provide agents by using film layers containing drug particles suspended in a polymer matrix.

Fine particles, whether nano- (e.g., submicron) or micro- (e.g., low micron) particles, can exhibit a strong tendency to agglomerate, which makes it difficult and expensive to obtain particles as free powders for processing into traditional compressed tablets. Now, the particles of the agglomerated agents can be suspended in a matrix and formed into a thin film sheet. In order to prevent further agglomeration, nanoparticles can be suspended in a stabilizing medium, such as a polymer or inorganic matrix. The agglomeration tendency of small drug particles traditionally has repercussions in the form of difficulty in producing uniformly distributed blends used for the compression of traditional tablets that predefines drug release characteristics. Now, placement of the small particles into a film prevents or diminishes agglomeration. The agglomerated particles can be useful and can be suspended in carrier film matrix and prepared into thin film sheets. The particle-containing sheets can be combined with various other sheets with specific functions in order to customize the delivery profile. Traditional solid dosage forms often make use of particle size reduction in order to increase the dissolution rate of poorly-soluble drugs. One problem is that the agglomeration tendency of fine particles has the effect of preventing the drug dissolution rate to be as fast as would be expected based solely on the particle size of the drug. The multilayer tablets of the instant invention can use films containing suspended particles of a drug as the drug carrier layer. This situation makes it possible for the multilayer tablets to contain fine particles in a configuration that permits such particles to be both spatially dispersed (i.e., non-agglomerated) and immobilized (i.e., non-agglomerating) in a polymer matrix.

FIG. 6 shows accelerated drug release rate of griseofulvin when drug particles are spatially dispersed and immobilized in a polymer film matrix. FIG. 7 shows that even at high load of drug particles in the film (˜72% w/w), the non-agglomerating effect of the film configuration is observed by the continued increase in dissolution rate with drug load. This shows the accelerated drug release of griseofulvin from the film matrix (HPMC or alginate) in comparison to the same type of particles (e.g., API), free from any polymer, occupying the same area of exposure to the solvent medium. The accelerated dissolution rate can be obtained with more than one type of polymer. FIG. 7 shows the dissolution rate profile for a range of drug loading percentages, where the drug release rate increases with drug load. However, at levels of about 72% (w/w) drug load in the film, the enhanced dissolution of drug from the immobilized particles is still observed. This is noteworthy because at the hypothetical limit of 100% drug load, the dissolution rate has to drop to the level of the bottom graph in FIG. 6.

The ability to tune specific film sheets for a specific function allows for control of release of agents from the ingestible unit. The versatility of using film sheets as a primary component for drug delivery systems is further demonstrated in FIGS. 8A-8B, where the control on the drug release rate afforded by the film configuration is shown. FIG. 8A shows the large change in drug release rate obtainable by adjusting the grade (e.g., molecular weight) of a HPMC polymer. Furthermore, this shows the tuning of dissolution rate obtained by mixing two different polymers in a film (e.g., HPMC and sodium alginate) in this example. As such, the specific film sheets can be configured alone or together to control drug release rate via selection of the polymer matrix for the film. FIG. 8A shows the effect of changing the molecular weight of the polymer while maintaining the same chemistry, and FIG. 8B shows the effect of using a mixed polymer matrix (e.g., HPMC/alginate) on drug release rate from the obtained film.

The embodiment of the invention that includes the thin sheets prepared with fine drug particles suspended and immobilized can be beneficial by making the process both easier and less expensive. It is easier and more versatile to work with particles suspended in films than with individual drug molecules dispersed in films. Furthermore, it is easier to work with particles immobilized in a dry film configuration than with dry particles in powder form. The use of particles can allow for “reformulation” to become faster and less expensive than conventional methods that cannot or do not use fine drug particles, or use fine particles in the form of powders. In traditional tablet formulation, when a faster or slower drug release is useful, a new formulation needs to be developed and tested, which is tedious and costly. On the other hand, the multifunctional film sheet ingestible unit makes “reformulation” a much faster and simpler process by allowing selection of specific sheets to provide specific functions. For example, the reformulation can be modified by changing the composition of the film sheets adjacent to a film sheet having a biologically active agent.

In one embodiment, the film sheets can be thin strips with an area of roughly 1 in2. The dimensions of such film sheets limit the total amount of drug that can be contained therein. For example, Zuplenz is commercially available in doses of 4 mg and 8 mg. This dosage amount is low compared to many drug products that need to be available in doses of 100 mg, 200 mg, or even greater. Now, the film sheets can be prepared with a specific or maximum amount of agent contained therein. The amount of dose needed can then be used to determine the number of film sheets in order to add up to the defined dose. For example, a multilayer ingestible unit with a drug load of 250 mg can be prepared by 50 sheets of a 5 mg film sheet.

In one example, tablets of griseofulvin containing 250 mg of the drug were prepared using the multilayer approach described herein. Also, 250 mg of a commercial product, Gris-PEG, was obtained in order to compare the drug release properties of the film sheet ingestible unit with the commercial product of griseofulvin. Films used in the fabrication of the multilayer tablets were made using the method described herein. The different “formulations” of multilayer tablets tested were obtained by stacking different layers of prefabricated films. Griseofulvin is a poorly-soluble drug, such that the drug content in the dosage form exceeds considerably the solubility of the drug in the dissolution medium. Therefore, the criterion for comparison among formulations focused on the time to attain a saturation concentration of the drug in the dissolution medium for a given formulation.

FIG. 9A includes a graph that illustrates an example where particle-containing films made from sodium alginate match or exceed the time to saturation of Gris-PEG. FIG. 9B includes a graph that shows that keeping the same polymer chemistry (e.g., Methocel K series) but changing the molecular weight is an effective method for modifying the drug release attributes of the multilayer ingestible units. Changing the molecular weight without changing the chemistry of the polymer, or the manufacturing process for the tablets, is a simple and effective way of modifying the drug release rate. FIG. 9C includes a graph that illustrates an example of the release profile when a layer of disintegrant is included.

One illustration of the versatility of this manufacturing method can include the number of different types of sheets that can be combined. In one example, one of the prefabricated film layers has the function of a disintegrant, such that it has the effect of breaking the multilayer ingestible unit into two or more pieces. The disintegration can occur immediately upon coming in contact with water, such as the stomach fluid, or delayed to occur in the small or large intestines. The disintegration can occur immediately upon coming in contact with saliva, producing a predefined breakage pattern into smaller pieces for easier swallowing. The result is that by addition of a functional disintegrant layer, the drug release can be accelerated without changing the polymer type, the molecular weight of the polymer, and/or the manufacturing method.

A fast-disintegrating multilayer tablet can be created by inclusion of one or more disintegrating functional layers. The number and placement of the disintegration layers relative to a drug layer can modulate the release profile.

FIG. 10 provides a graphical synopsis of the versatility offered by the multilayer film-based tablets in terms of the ability to control drug release, where the overall drug release rate at the 30 min time point in the dissolution test is presented. The multilayer tablet method makes it possible to use the same basic manufacturing procedure to produce by assembling tablets with a wide range of drug release properties. Depending on the selection of the thin sheet, the multilayer tablets can be made to have a predetermined release rate. Thus, the ingestible unit can include a drug release rate that is the same as an existing dosage form or be configured with different film sheets so that the release rate is faster or slower than an existing product. The multilayer film tablets allow for modifying or customizing patent-specific drug release properties by designing a tablet to include a specific arrangement of film sheets. This makes it possible to replace one sheet for another to modify a dosage format.

The prefabricated multilayer film ingestible units of tablets or capsules have numerous favorable applications. The functionalities can be tailored to match certain medical arenas or particular needs for certain therapies or certain nutritional supplements, such as is the case of pediatric and geriatric formulations. The pharmacokinetics and metabolism of drugs in pediatric patients are quite often different from those of the adult population, for which most existing pharmaceutical products are typically developed. The multilayer film ingestible units offer flexibility for customizing dose and drug release profiles to needs or requirements. Multilayer film ingestible units can be tailored to make it possible to design a composite drug release profile using layers with different release characteristics in order to match the release profile requirement for a pediatric formulation.

FIG. 11 provides a graph that shows the amount of griseofulvin released as a function of time for different molecular weights of methylcellulose carriers as well as model predictions. The multilayered units can be designed based on models, wherein modeling drug release from individual layers allows for determining the layers for multilayered dosage forms. FIG. 11 shows the changes with constant drug load and different polymers. On the other hand, FIG. 12 shows the same polymer with different drug loads. As such, the individual sheet drug release profiles can be tailored and combined to provide an overall multilayered tablet release profile.

FIG. 5A provides a graph that shows the amount of dissolved griseofulvin from HPMC films and alginate films loaded at 9.3% compared to pure drug films. This shows the effect of film configuration on dissolution rate. FIG. 5B provides a graph that shows the percent of dissolved griseofulvin from HMPC films with agglomerated particles compared to HMCP films without any appreciable agglomerated particles from 9.3% loadings. This shows that particles can be used when loaded or suspended in a thin film sheet matrix, and the effect on dissolution rate. FIG. 5C provides a graph that shows the percent of dissolved griseofulvin from HMPC and/or SA LVCR films with 9.3% griseofulvin. This shows the effect of polymer type and polymer combination. FIG. 5D provides a graph that shows the percent of dissolved griseofulvin as a function of film thickness, where increasing film thickness from 10 microns to 20 microns can initially increase the dissolution profile, but increasing up to 70 microns significantly decreases the dissolution profile. A 10 micron film can be configured with the film is a single layer of particles that are held together by the polymer sheet, where the particles may or may not be larger in diameter than the film thickness. In one example, a films can be on the order of 100 microns. However, the film can be as thin as roughly the size of the largest particles. The film can also be much thicker than the average diameter of the particles as shown in FIGS. 3-4.

In one embodiment, the present invention can include an ingestible unit that has a plurality of discrete sheets that are combined into an ingestible unit. Each of the discrete sheets can have a composition configured for one or more predetermined functions. These functions can be combined to obtain each function or a synergistic combined function. The unit can be designed by selecting different functions, and using discrete sheets having that function. The function can range from providing a drug, to modulating drug release, to unit identification and anti-counterfeiting. For example, the one or more predetermined functions for each discrete sheet are selected from the functions described herein or known to one of ordinary skill in the art.

In one embodiment, the ingestible unit can be configured to include at least one discrete sheet of a first type, and at least one discrete sheet of a second type different from the first type. In one example, at least one discrete sheet of the first type includes a substance that is devoid in the at least one discrete sheet of the second type. In another example, at least one discrete sheet of the first type is different from the at least one discrete sheet of the second type. The difference from the first type to the second type can be in a characteristic selected from the group consisting of: composition of sheet; type of filler; type or amount of particles in sheet; size of particles in sheet; distribution of particle sizes in sheet; type or amount of a biologically active agent in sheet; bioactive agent combination in sheet; rate of bioactive agent release from sheet; mechanical strength of sheet; flexibility of sheet; rigidity of sheet; color of sheet; radiotranslucency of sheet; radiopaquness of sheet; identifying substance in sheet; anti-counterfeiting substance in sheet; type or amount of polymer in sheet; type or amount of inorganic substance in sheet; dimension of sheet; structure of sheet; water-dissolvability of sheet; water-stability of sheet; type or amount of a film agent in sheet; type or amount of a plasticizer in sheet; type or amount of a taste-masking agent in sheet; type or amount of a coloring agent in sheet; type or amount of a solubilizing agent in sheet; type or amount of an effervescent agent in sheet; type or amount of an antioxidant in sheet; type or amount of an absorption enhancer in sheet; type or amount of a disintegrating agent in sheet; type or amount of a pH buffer agent in sheet; type or amount of a surfactant in sheet; type or amount of a complexing agent in sheet; type or amount of a bioadhesive agent in sheet; type or amount of a sheet adhesive in sheet; type or amount of osmotic agent in sheet; type or amount of identifying agent in sheet; type or amount of anti-counterfeiting agent in sheet; type or amount of a tracking agent in sheet; type or amount of transporter inhibitor; type or amount of transporter inducer; type or amount of emulsifying agent, type or amount of self-emulsifying system; type or amount of crystallization inhibitor; type or amount of crystallization promoter; type or amount of supersaturation promoting agent; type or amount of antimicrobial preservative; type or amount of catalyst; type or amount of chelating agent; type or amount of particles; type or amount of organoleptic agent, such as a flavor and/or scent agent; type or amount of identifying device; type or amount of anti-counterfeiting device; and/or type or amount of anti-counterfeiting pattern. Optionally, the ingestible unit can include at least one discrete sheet of a third type different from the first type and second type. Also, any number of different types of sheets can be used. The different characteristics can provide the different functions of the sheets described herein. The different characteristics can be obtained by changing the type or amount of ingredients in a sheet.

In one embodiment, each of the plurality of discrete sheets is substantially identical. There may be some instances where it can be advantageous to combine a plurality of the same sheets to arrive at the desired ingestible unit. For example, a single sheet may be a suitable carrier matrix, but not hold enough drug, so a number of the film sheets are combined in order to arrive at the desired drug dose.

In one embodiment, one or more of the discrete sheets are water-dissolvable. These water-dissolvable sheets can be configured for dissolving, breaking or disintegrating in the mouth, after swallowing, in the stomach, or in the intestines. The water-dissolving sheets can be placed at a location where it is desirable to open a laminated body to expose a drug sheet, effervescent sheet, or the like. The dissolving sheets can break the laminated body into a number of thin sheet bodies.

In one embodiment, one or more discrete sheets are water-stable. It can be advantageous to have some of the film sheets be stable and not dissolvable in the mouth or stomach. In some instances, it can be desirable to have film sheets that are completely water-stable. For example, a coating can be provided that is substantially water-stable to delay dissolution. Water-stable sheets may still be useful for releasing biologically active agents.

In one embodiment, the discrete sheets can be prepared from any type of film or laminate-forming material. That is, any material that can be formed into a thin film can be used as a sheet having a particular function of the present invention. The film-forming material can range from polymers that are natural or synthetic. However, the film-forming material may be a substance other than a polymer. In one example, the film-forming material can be calcium phosphate, talc, calcium silicate, calcium carbonate, derivatives thereof, and combinations thereof, where calcium embodiments may be formed into laminates. In one example, the film-forming material can be a carrageenan.

In one embodiment, the one or more discrete sheets can be formed from a polymer. The polymer can be any type of polymer that forms a thin sheet, such as a film. The polymer can be natural, such as a polysaccharide or synthetic. There are a large number of different types of polymers that can be used. Any edible, biocompatible, or generally-recognized-as-safe polymer can be used. Some non-limiting examples can include polymers selected from the group consisting of gelatin, hydroxyethyl cellulose, cellulose acetate phthalate, hydroxypropyl methylcellulose phthalate, carboxymethyl ethylcellulose, hydroxypropyl methylcellulose acetate succinate, polyvinyl acetate phthalate, maltodextrin, dextran, hydroxypropyl cellulose, sodium carboxymethyl cellulose, poly(methacrylic acid-co-ethyl acrylate), poly(methacrylic acid-co-methyl methacrylate), polyvinylpyrrolidone, polylactic acid (PLA), poly-L-lactide (PLLA), poly-D-lactide (PLDA), poly(lactic-co-glycolic acid) (PLGA), polysaccharides, Soluplus®, derivatives thereof, and combinations thereof. In one aspect, the polymer can be a polysaccharide, such as a polysaccharide selected from the group consisting of methylcellulose, hydroxypropylmethylcellulose, ethylcellulose, sodium alginate, starch, chitosan, chitin, pullulan, agar, derivatives thereof, and combinations thereof.

In one embodiment, the one or more discrete sheets can be formed from a film forming protein such as gelatin or zein protein.

In one embodiment, the one or more discrete sheets can be formed from an inorganic component. The inorganic component can be prepared into a laminate sheet. Some non-limiting examples of inorganic components that can be prepared into the discrete sheets include calcium phosphate, calcium silicate, calcium carbonate, derivatives thereof, and combinations thereof. Some inorganic materials can be formed into ceramic thin films or laminates.

The discrete sheets can have various configurations and formats as long as each sheet can be prepared into the ingestible unit described herein. The sheets have a larger width and length compared to thickness. This provides a top surface and a bottom surface with a significant cross-sectional profile, but is relatively thin in order to be a sheet. The sheets can be films, lamina, laminates, wafers, or other thin-body structures. The films can range from about 100 nanometers to many microns in thickness. A sheet can be a lamina or a laminate of a plurality of lamina. The lamina can be a layer that is combined with other lamina layers into a laminate. While the discrete sheets are described to be combined into laminate structures, the individual sheets themselves can be prepared as laminates from one or more types of thin lamina. The thin lamina can be a thin plate or layer of material, which often is of a sub-micron size. The lamina can be prepared into a laminate and used as a sheet. The sheet can also be prepared as a wafer, which can be a thin member, such as a thin disk, and may be a dried paste, gelatin adhesive paper, or the like. Also, the wafer may be a thin sheet enclosing or containing a powder or particles, where the powder or particles can be a biologically active agent.

In one embodiment, a plurality of discrete sheets of the ingestible unit can be laminated together. The discrete sheets can be random or arranged with respect to the laminated body. When random, the different sheets to be combined can be arranged without any particular order, and then laminated together so that the sheets are in a random sequence from one surface to the opposite surface. However, it can be advantageous to arrange the sheets in a desired order. For example, it may be beneficial to arrange solubilizing sheets or disintegrating sheets at a relative position with respect to a sheet containing a biologically active agent, and thereby predetermining an arrangement can allow for enhanced control over release of the biologically active agent. The sheets can be arranged in order to provide a controlled release profile in order to increase or decrease the rate of agent dissolution from the laminated body. Also, the selected arrangement can allow for disintegrating sheets to be placed at specific locations in order to allow the laminated body to break into thinner laminated bodies upon disintegrating. The sheet layers on opposite sides or between the disintegrating sheets can break away from each other upon disintegration. Also, the sheets can be arranged in a manner that allows for the staggered release of agents from one or more sheets before agents (e.g., the same agent or different agent) are released from one or more other sheets. As such, the sheets can be arranged in a sequential order for sequential release of active agent from the laminated body. Also, non-dissolving or slow-dissolving sheets can be included at ends or the surfaces of the laminated body in order to inhibit dissolution or agent release until the unit is in the stomach, large intestine, or small intestine.

When the sheets are laminated together, these sheets may or may not be adhered together with an adhesive. In one aspect, a sheet adhesive agent can be used to adhere adjacent discrete sheets together. In one aspect, adjacent sheets of the plurality of discrete sheets can include polymers that adhere together and adhere the adjacent discrete sheets together. In one aspect, adjacent discrete sheets of the plurality of discrete sheets can include sheet adhesive agents that adhere the adjacent discrete sheets together.

In one embodiment, the one or more discrete sheets can be separate from one or more other discrete sheets. That is, one or more of the discrete sheets can be separate from other discrete sheets, or not laminated or otherwise adhered to other sheets. One or more of the discrete sheets can be of different dimensions than the majority of the others, creating inter-sheet voids for capillary uptake of fluids. Also, stacking of different sized sheets can provide for rounded edges on the ingestible unit. As such, some or all of the sheets may be loose within an ingestible container. While some of the sheets may be laminated or otherwise adhered together, one or more of the discrete sheets can be loose or not attached to the other sheets. The loose sheets can be combined together in a container that can be consumed as an ingestible unit. Such an ingestible unit can include the sheets loosely packaged together or packaged as a stack of sheets that are not adhered or attached together. A capsule or other similar type of container can be used for the ingestible container having the plurality of discrete sheets to be ingested. The sheets in the capsule can be random or arranged in an order. For example, the sheets can be dimensioned so that they flow freely in the capsule or are in a random arrangement in the capsule. In another example, the sheets can be dimensioned so that they can be stacked and retained in the stack within the capsule, which can include the sheets being cut into a circular cross-section and stacked into a cylindrical shape before being encapsulated. The non-adhered stacked sheets can also be encapsulated within a coating in order to provide a unit with stacked sheets that are not laminated together. However, any coating can also coat a stack of laminated sheets, and laminated sheets may be included in a capsule or other edible container. The coating or encapsulated sheets may include a combination of laminated and non-laminated or loose sheets. When a coating is used, it can be any type of pharmaceutical coating, such as those known in the art, or any ingestible coating, such as a polymeric coating or other film coating. The coating may also include a biologically active agent. The coating can be a thin film that is formed to encapsulate the discrete sheets.

In one embodiment, the ingestible unit can be configured as any ingestible unit that is chewable and/or swallowable or otherwise orally-administrable. Preferably, the unit is consumed without chewing by chasing the unit with water. As such, the unit can be configured with a dimension similar to any swallowable medicament or solid dosage form. The unit can be shaped and/or sized as any tablet, sprinkle, caplet, capsule, or the like. However, the ingestible unit may also be administered rectally or vaginally, such as by being configured as a suppository. The units described herein can be modified or designed as a suppository.

In one embodiment, the ingestible unit is configured as a placebo. That is, the unit is devoid of a specific bioactive agent. The placebo configuration can be prepared to appear identical to a unit that contains a biologically active agent, and both the placebo and biologically active agent ingestible units can be used in studies, such as clinical trials. The placebo embodiment may include one or more active agents, but omit a specific active agent that is included in a different unit. The placebo may be designed to test the activity of an agent by being devoid of that agent, such as by being used in a blind clinical trial with another unit that has the agent. The placebo embodiment may be included in a kit that includes a biologically active agent-containing embodiment. The placebo embodiment may be devoid of any active agents, and may include one or more non-drug sheets that are included in a biologically active agent-containing embodiment.

The ingestible unit can include one or more sheets with one or more distinct components. Each sheet can be different in the ingestible unit, or two or more sheets may be the same or at least include the same components. The types or amounts of the components can vary between sheets. The one or more components that can be included in the sheets can include the following: a film-forming agent; a filler; a plasticizer; a taste-masking agent; a coloring agent; a solubilizing agent; an effervescent agent; an antioxidant; an absorption enhancer; a disintegrating agent; a pH modifying or buffer agent; a surfactant; a complexing agent; a bioadhesive agent; a sheet adhesive; an identifying agent; an anti-counterfeiting agent; a tracking agent; transporter inhibitor agent; transporter inducer agent; emulsifying agent, self-emulsifying system agents; crystallization inhibitor; crystallization promoter; supersaturation promoting agent; antimicrobial preservative; catalyst; chelating agent; particles; organoleptic agent; flavoring agent; scent agent; identifying device; and/or anti-counterfeiting device. These types of ingredients can be exemplified by substances that are commonly used for pharmaceutical compositions or other ingestible compositions. Preferably, these types of ingredients are defined as generally recognized as being safe (GRAS) by a government agency, such as the U.S. FDA. In one embodiment, the ingredients can be defined as being approved by a select committee on GRAS substances (SCOGS), such as which can be found at the U.S. FDA website, which is incorporated herein by specific reference in its entirety, specifically included is the GRAS and SCOGS ingredients.

The film agent can be selected from methylcellulose, hydroxypropylmethylcellulose, ethylcellulose, sodium alginate, poly(methacrylic acid-co-ethyl acrylate), poly(methacrylic acid-co-methyl methacrylate), starch, polyvinylpyrrolidone, polylactic acid (PLA), poly-L-lactide (PLLA), poly-D-lactide (PLDA), poly(lactic-co-glycolic acid) (PLGA), chitosan, chitin, pullulan, derivatives thereof, and combinations thereof, or the like. The plasticizer can be selected from glycerine, triacetin, triacetyl citrate, polyethyleneglycol, mineral oil, myglyol, derivatives thereof, and combinations thereof, or the like. The taste-masking agent can be selected from kleptose, cyclodextrin, cyclodextrin derivatives, ginger, anise, cinnamon, peppermint, licorice, fruit flavoring, citric acid, fruit juice, sweeteners, sucrose, glucose, fructose, mannitol, saccharin, aspartame, sucralose, Stevia plant derivatives, honey, derivatives thereof, and combinations thereof, or the like. The coloring agent can be a food colorant, such as carotenoid compounds and FD&C red, green, yellow, and blue, or the like. The solubilizing agent can be selected from polyvynilpyrrolidone, polyvinylcaprolactam-polyvinylacetate-polyethyleneglycol copolymer, fatty acids, castor oil, cyclodextrins, polyethyleneglycol, glyceryl distearate, lecithin, monoglycerides, diglycerides, triglycerides, propylene glycol monostearate, Labrafils (e.g., oleoyl macrogol-6 glycerides, oleoyl polyoxyl-6-glycerides, linoleoyl macrogol-6 glycerides, linoleoyl polyoxyl-6 glycerides, lauroyl macrogol-6 glycerides, lauroyl polyoxyl-6 glycerides), Labrasols (e.g., caprylocaproyl macrogol-8 glycerides, caprylocaproyl polyoxyl-8 glycerides), Solutols (e.g., poly-oxyethylene esters of 12-hydroxystearic acid), Soluplus (e.g., polyvinyl caprolactam-polyvinyl acetate-polyethylene glycol graft co-polymer), derivatives thereof, and combinations thereof, or the like. Soluplus can also be used as a film-forming agent. The effervescent agent can be selected from sodium carbonate, bicarbonate, potassium carbonate, calcium carbonate, citric acid, malic acid, tartaric acid, adipic acid, fumaric acid, derivatives thereof, and combinations thereof, or the like. The antioxidant can be selected from tocopherol, vitamin E, resveratrol, ascorbyl palmitate, tert-butylhydroquinone, resveratrol, nordihydroguaiaretic acid, cysteine, propyl gallate, octyl gallate, 3-tert-butyl-4-hydroxyanisole, butylated hydroxytoluene, ascorbic acid, derivatives thereof, and combinations thereof, or the like. The absorption enhancer can be selected from fatty acids, chitosan, sodium caprate, sodium deoxycholate, dipotassium glycyrrhizinate, furanocoumarins and grapefruit derivatives, bile salts, ethylenediaminetetraacetic acid, tocopheryl polyethyleneglycol succinate (TPGS), derivatives thereof, and combinations thereof, or the like. The disintegrating agent can be selected from croscarmellose sodium, sodium starch glycolate, insoluble polyvinylpyrrolidone, carboxymethylcellulose, derivatives thereof, and combinations thereof, or the like. The pH modifier or buffer agent can be selected from sodium carbonate, magnesium carbonate, calcium carbonate, sodium hydroxide, potassium hydroxide, ascorbic acid, citric acid, succinic acid, fumaric acid, derivatives thereof, and combinations thereof, or the like. The surfactant can be selected from sodium lauryl sulfate, poloxamers, sorbitan esters, polysorbates, sorbitans, stearic acid, derivatives thereof, and combinations thereof, or the like. The complexing agent can be selected from cyclodextrins, calcium glycerophosphate, dodecyl 2-(N,N-dimethylamino) propionate, zinc, dextran, pectin, copper acetate, sodium deoxycholate, calcium, magnesium, derivatives thereof, and combinations thereof, or the like. The bioadhesive agent can be selected from gelatin, starch, glycoproteins, proteins, carbohydrates, mucopolysaccharides, derivatives thereof, and combinations thereof, or the like. The sheet adhesive can be selected from polyvinylpyrrolidone, polyvinyl alcohol, polyvinyl acetate, confectionary glue, starch, derivatives thereof, or combinations thereof, or the like. The tracking agent, identifying agent, or anti-counterfeiting agent can be selected from fluorescein, rhodamine, succinimidyl esters, maleimide activated fluorophores, fluorescent dyes, fluorescent particles, infrared active particles, near infrared active particles, metallic nanoparticles, polymeric particles, silica based nanoparticles, SERS (Surface Enhanced Raman Spectroscopy) particles, raman active particles, derivatives thereof, and combinations thereof, or the like. The osmotic agent can be selected from mannitol, osmitrol, dextrose, sucrose, fructose, sodium chloride, potassium chloride, xylitol, sorbitol, lactose, potassium phosphate, derivatives thereof, or combinations thereof, or the like. The transporter inhibitor can be selected from elacridar, zosuquidar, glibenclamide, quinaxoline derivatives, phenylalanine, arginyl □-naphthylamide, grapefruit derivatives, furanocoumarins, derivatives thereof, and combinations thereof, or the like. The transporter inducer can be selected from xenobiotics, diallyl sulfide, dexamethasone, derivatives thereof, and combinations thereof, or the like. The emulsifying agent can be selected from tocopheryl polyethyleneglycol succinate (TPGS), Cremophor (e.g., non-ionic polyethoxylated detergents), Lutrol (e.g., polyethylene glycol), Poloxamers (e.g., polyethylene-polypropylene glycol), cholesterol, octyldodecanol, polyoxylglycerides, derivatives thereof, and combinations thereof, or the like. The self-emulsifying system can be selected from Labrasol, Labrafil, Cremophor, Pluronics, Lutrol, poloxamers, polysorbates, ethyl linoleate, mono- and diglycerides of capric and caprylic acids, tocopherol acetate, Solutol, soybean oil, tocopheryl polyethyleneglycol succinate (TPGS), Capmuls, derivatives thereof, and combinations thereof, or the like. The crystallization inhibitor can be selected from polyvinylpyrollidone, hydroxypropylmethylcellulose, silicon dioxide, dextrins, dextrans, bile acids, sterols, polysebacic anhydride, derivatives thereof, and combinations thereof, or the like. The supersaturating promoting agent can be selected from hydroxyproylmethylcellulose, hydroxypropylmethylcellulose acetate succinate, polyvinylpyrollidone, derivatives thereof, and combinations thereof, or the like. The antimicrobial preservative can be selected from benzoic acid, sodium benzoate, methyl paraben, propyl baraben, butyl paraben, sorbic acid, propionic acid, dehydroacetic acid, derivatives thereof, and combinations thereof, or the like. The catalyst can be selected be heavy metals selected from Ni, Cr, Mn, Zn, Fe, or combinations thereof, or the like. The organoleptic agent can be a flavorant or scent, such as selected from vanilla, bubble gum, fruit flavor, mint, chocolate, licorice, marshmallow, peanut butter, aspartame, sucralose, sucrose, glucose, citric acid, stevia plant, derivatives thereof, or combinations thereof, or the like. The organoleptic agent for a veterinary embodiment can be selected from glutamates, chicken flavor, umami flavoring, beef flavor, fish flavor, or the like. The chelating agent can be selected from disodium edetate, EDTA, pentetic acid, derivatives thereof and combinations thereof, or the like.

The functional sheets can have the following compositions: a biologically active agent sheet can include the agent, a film-forming agent, and a plasticizer; a solubilizing sheet can include a film-forming agent, plasticizer, and solubilizing agent; an effervescent sheet can include a film-forming agent, plasticizer, and effervescent agent; an antioxidant sheet can include a film-forming agent, plasticizer, and antioxidant; a taste-masking sheet can include a film-forming agent, plasticizer, and taste-masking agent; a coloring sheet can include a film-forming agent, plasticizer, and coloring agent; an absorption enhancing sheet can include a film-forming agent, plasticizer, and absorption enhancing agent; a pH-modifier or buffer sheet can include a film-forming agent, plasticizer, and pH modifying agent; a disintegrating sheet can include a film-forming agent, plasticizer, and disintegrating agent; a complexing sheet can include a film-forming agent, plasticizer, and complexing agent; an identification or anti-counterfeiting sheet can include a film-forming agent, plasticizer, and identification or anti-counterfeiting agent; and a surfactant sheet can include a film-forming agent, plasticizer, and a surfactant agent.

The ingestible unit can be configured to include one or more biologically active agents, which can be dissolved or suspended in one or more of the discrete sheets. The one or more sheets having the biologically active agent can be located at various locations within the laminated body embodiment. In one example, at least one discrete sheet has a biologically active agent, which sheet is embedded within a laminated body between other discrete sheets. That is, the sheet having the biologically active agent can be located between the two opposite surface sheets. Alternatively, at least one sheet having a biologically active agent can be located on an external surface of a laminated body. In one aspect, the ingestible unit can include at least two discrete sheets each having a different biologically active agent. As such, the ingestible unit can provide at least two different biologically active agents. One or more layers may be located between the different biologically active agent sheets. The different biologically active agent sheets can be surrounded by different sheets that control the release profile of each of the biologically active agents. For example, one biologically active agent can be configured for complete release before a second biologically active agent is released. In another example, two or more different layers of the same drug can be configured for different release profiles where one releases before the others, which can provide an extended, bimodal or multi-modal release profile. Any number of combinations of biologically active agents may be combined into a single ingestible unit, where sheets having different biologically active agents can be adjacent or separated by one or more other sheets.

The ingestible unit can have various numbers of discrete sheets, usually more than one, and often more than two. The number of discrete sheets for most applications, such as drug delivery, can be between about 5 to about 500 sheets, from about 6 to about 400 sheets, from about 7 to about 300 sheets, from about 8 to about 200 sheets, from about 9 to about 150 sheets, or from about 10 to about 100 sheets. Pediatric ingestible units often have smaller drug requirements, and may be prepared with less than or about 10 sheets, such as from about 3 to about 9 sheets, about 4 to about 8 sheets, about 5 to about 7 sheets, or about 6 sheets. However, in most drug-containing ingestible units can include from 10 to 100 sheets, from about 20 to about 90 sheets, from about 30 to about 80 sheets, from about 40 to about 70 sheets, or from about 50 to about 60 sheets.

The discrete sheets can have various thicknesses depending on the ingredients and matrix materials. The thickness of a sheet can range from about 100 nm to about 500 microns. Nano-scale sheets can range from about 100 nm to about 1000 nm, from about 200 nm to about 900 nm, from about 300 nm to about 800 nm, from about 400 nm to about 700 nm, or from about 500 to about 600 nm. The micron-scale sheets can range from about 1 micron to about 500 microns, from about 10 microns to about 250 microns, from about 20 microns to about 200 microns, from about 30 microns to about 150 microns, from about 40 microns to about 125 microns, from about 50 microns to about 100 microns, from about 60 microns to about 90 microns, or from about 70 microns to about 80 microns. However, it should be recognized that the sheets can have any thickness that allows for preparation into an ingestible unit as described herein. In one example, each discrete sheet has a thickness less than 50 microns.

The size of ingestible unit may also vary, but generally is in the size range of common pharmaceutical dosage forms. Generally, most dosage forms are less than 25 mm in the longest dimension, and are often cylindrical with a smaller dimension in the diameter or length. The largest dimension can be from one sheet to an opposite sheet, or the largest dimension can be the diameter or width of a sheet. Dosage forms can greatly vary in size and shape as is common in the pharmaceutical industry. The largest dimension of the ingestible unit can be less than or about 50 mm, often less than 40 mm, which can be exemplified by less than 25 mm, less than or about 20 mm, less than or about 15 mm, less than or about 10 mm, less than or about 5 mm, less than or about 2.5 mm, or less than or about 2 mm. In one aspect, the largest dimension can be defined by the summation of the thicknesses of the discrete sheets. In one example, the ingestible unit can be cylindrical with a length of 25 mm and a diameter of 7 mm. In one aspect, larger ingestible units can be prepared for animal health products that include biologically active agents for use in animals. Additionally, significantly larger units can be used for the agricultural, pesticidal, herbicidal, or fertilizer embodiments.

In one embodiment, the biologically active agent or other agent, such as an identification agent or anti-counterfeiting agent can be present in a particle. As described herein, the particle can be an agglomeration of agent molecule with or without an additional agglomerating agent. The size of the particles can vary. Generally, the size of the particles can be smaller than the thickness of the discrete sheets as shown in FIGS. 3-4. The particles can be the same size as the nano-scale or micron-scale as provided herein.

The ingestible unit can have any number of combinations of the different discrete sheets that are described herein. However, one embodiment of the ingestible unit includes at least one discrete sheet having a biologically active agent, at least one discrete sheet having a solubilizing agent, and at least one discrete sheet having a disintegrating agent.

The ingestible units can be prepared by a number of different methodologies depending on whether the discrete sheets are loose in an edible container or laminated together. The discrete sheets can be manufactured as needed, or they may be prepared elsewhere and supplied to the manufacturer to make the ingestible unit. Accordingly, a method of making an ingestible unit described herein can include obtaining the discrete sheets and combining the discrete sheets into the ingestible unit. Since the ingestible units are prepared to have one or more specific functions, the method can also include determining the predetermined functions for each discrete sheet. The sheets that are included can be selected based on the function provided, and thereby a combination of functions can be implemented into a single ingestible unit.

The individual sheets can be selected for the ingestible unit based on their individual functions. As such, the method can include determining a characteristic that can be different between a first type of discrete sheet and a second type of discrete sheet. That is, a characteristic can be determined to be present in one sheet, but absent in another. For example, one sheet can have a drug while another sheet is devoid of the drug. Alternatively, the characteristic can be determined to be different between two sheets, such as by a sheet being selected that has a certain drug amount and another sheet can have the same drug at a different amount. Also, a first sheet can have a specific list of ingredients at certain concentrations, and a different sheet can have the same specific list of ingredients with one or more being at different concentrations from the first sheet.

In one embodiment, the present invention can include preparing each discrete sheet before including each sheet in the ingestible unit. The sheets can be prepared as massive sheets or continuous extrudates and then cut to size and/or shape. Alternatively, the sheets can be formed into a selected size and/or shape. The one or more of the discrete sheets can be prepared to be films, lamina, laminates, or wafers as described herein.

In one embodiment, the polymeric film embodiment of the discrete sheets can be prepared by common methods of preparing polymer films. For example, the film sheets can be prepared by casting, spin coating, extrusion and calendering, roll-pressing, microfabrication, molding, jet-printing, drop-on demand printing, dot printing, or any other useful process.

In one embodiment, the discrete sheets can be prepared from inorganic materials. As such, the discrete sheets of inorganic materials can be prepared by roll pressing.

In one embodiment, the ingestible unit can be prepared to have a specific amount of biologically active agent, such as a specific amount of drug. The amount of drug included should be consistent across a number of batches so that the ingestible unit can achieve regulatory compliance. Accordingly, the method of preparing an ingestible unit can include: preparing a composition having a biologically active agent at a defined amount; forming the composition into a discrete sheet to have the biologically active agent at a defined amount; and including the discrete sheet in the ingestible unit. The method may also include: determining a dose of a biologically active agent to be included in one or more discrete sheets of the ingestible unit; determining an amount of the biologically active agent in a discrete sheet; determining a number of discrete sheets having the biologically active agent to combine to obtain the determined dose; and combining the number of discrete sheets having the biologically active agent in the ingestible unit. An accurate dose can be obtained by characterizing the amount of drug in a single sheet of a specific size, and then combining the sheets in order to obtain the desired dosage. Drug loading into sheets can be up to about 70% by weight of the sheet, and often up to about 50% by weight. In one example, 27 sheets can be used that have drug. The amount of drug in each sheet can be calculated before or after shaping the individual sheets or ingestible unit into the size and shape of the dosage form. The amount of drug lost during processing can also be taken into account in order to design the ingestible units and select the appropriate number of sheets to arrive at the predetermined dose. However, it should be recognized that a single sheet may be suitable for the entire dose of an ingestible unit. The same methodologies can be performed when the ingestible unit includes two or more different biologically active agents. Moreover, this methodology can be used for determining the amount of any agent or ingredient described herein.

In one embodiment, an agent, such as biologically active agent or anti-counterfeiting agent, can be included as particles in the discrete sheets. The particles can be embedded entirely within the sheet matrix or embedded in a surface to provide a rough profile. Some particles may be exposed in the surface. The particles can be obtained or prepared by aggregating a number of agent molecules together. The size of the particles can vary or be uniform. Preferred particle sizes can range from about 10 nm to about 1000 microns, from about 1 micron to about 50 microns, and from about 10 microns to about 100 microns. Also, the sheets having the particles can be prepared on-site or obtained from a supplier. The sheets can be made by: preparing a plurality of particles having a biologically active agent; suspending the plurality of particles in a composition; and forming the composition into a discrete sheet. The sheets can then be included in the ingestible unit. The sheets can include a high load of particles, which can be about 50% by weight or +/−10% or 20%. This can allow for the ingestible unit to include as much drug as current tablets or capsules, which can be over 1000 mg in some instances, but usually containing low hundreds of milligrams. The sheets can include a low load of particles, which can be about 5% to 1% by weight. The sheets can include a very low of particles, which can be about 1% or less.

In one embodiment, the ingestible unit can be prepared by encasing the sheets in an ingestible container to form the ingestible unit. The sheets can be encased in the container while loose or laminated together. The sheets can be sized and shaped to fit tight with an internal surface of an ingestible container, or they may be smaller so that the sheets can move around in the container, and a plurality of sheets may be flowable in the container. The container often can be a capsule, but other formats may be useful.

In one embodiment, the ingestible unit can be prepared by stacking the plurality of discrete sheets. The stacked sheet may or may not be laminated together. In some instances the stacked sheets can be adhered with each other to form a laminated body. In others, the stacked sheets may be loose and packed into a correspondingly-sized ingestible container. In some instances, both sets of adhered and sets of loose sheets can be stacked. While the order of the sheets in a stack may be random, the unit can be designed to have a specific order of stacked sheets, which may be arranged in the determined order from a first side to opposite second side.

In one embodiment, the ingestible unit can be prepared by shaping the sheets or a stack of sheets. That is, the individual shapes may be shaped and then combined loosely or stacked, or the stacked sheets can be shaped. The shaping can be by any method, such as laser, stamping, cutting, or the like. In one example, the sheets and/or stack are shaped so as to be devoid of a sharp corner. The stacked sheets may stick together by having the carrier matrices prepared from sticky materials. Also, pressing can cause the sheets to stick together. The stacked sheets may also be coupled together by using a belt or a band that wraps around the sheets and cinches the sheets together. The belt or band can be ingestible, such as by being a gelatin similar to the capsules. Also, the stacked sheets may be riveted together.

In one embodiment, a method of designing or preparing an ingestible unit can include: identifying a predetermined function for each of the discrete sheets; and preparing the discrete sheets to have the predetermined function. The method can include selecting two or more predetermined functions to be combined into an ingestible unit, and combining discrete sheets having the one or more predetermined functions into the ingestible unit. The method can include determining an arrangement of the discrete sheets having the one or more predetermined functions, and arranging the discrete sheets into the determined arrangement.

In one embodiment, an ingestible unit can include a sheet that is configured with an identification agent or anti-counterfeiting agent. The anti-counterfeiting agent can be placed on a sheet according to a specified pattern. However, the ingestible unit can include a plurality of sheets with identification agent or anti-counterfeiting agent arranged themselves in a pattern. The individual sheets can have specific patterns or the sheets can be arranged in specific patters. The pattern of these sheets in the unit can be used for identification or authentication similar to a bar-code or fingerprint. Different types of ingestible units can have different patterns.

In one embodiment, the present invention can include a method of administering an ingestible unit to a subject. The administration can be performed to either intend to provide a therapeutic effect or provide a placebo. The ingestible unit having a biologically active agent, such as a drug, can be administered in a therapeutically-effective amount to inhibit or treat a disease. The disease to be treated can determine the drug and dosing parameters, which can be easily determined with the teachings provided herein. The therapy can be provided by one or more drugs. For example, a drug combination can include rifampicin and isoniazid in separate sheets with different release rate profiles, where rifampicin is released in the stomach and isoniazid is released in the small intestine.

In one embodiment, an ingestible unit can be designed to have multiple functions. As such, an existing drug formulation or design can be analyzed for function of formulation components, and these identified functions can be incorporated into a sheet by having the same components or similar-functioning components. This can include preparing a blueprint of functionalities or component types to be included in a sheet or in the multi-sheet ingestible unit. A drug formulation ingestible unit can be changed by changing one or more of the functional sheets and optionally reusing other functional sheets. For example, the molecular weight of a film-forming agent can be increased in order to reduce the rate of release of an agent (e.g., drug or anti-counterfeiting) from the sheet.

In one embodiment, one or more of the ingestible units can be included in any food product, such as ice cream, yogurt, or the like.

In one embodiment, the biologically active agent can be a nutritional supplement, such as iron, which is difficult to administer with proper absorption. Iron is soluble in acidic pH, and usually precipitates in the intestine at neutral pH. Now, one or more pH-modifying sheets that produce an acidic environment or microenvironment can be included with the iron sheet. Similarly, other specific functionalities can be provided to improve the absorption of biologically active agents. Also, complexing and/or chelating agents can be included in sheets to complex with agents to facilitate absorption from the intestine.

In one example, an average thickness of a sheet is about 100 microns+/−about 20 microns. The thickness of an ingestible unit can be from about 3 to about 40 sheets.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally-equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can, of course, vary. It is also to be understood that the terminology used herein is for the purpose of describing particular embodiments only, and is not intended to be limiting. All references recited herein are incorporated herein by specific reference in their entirety.

1. An ingestible unit comprising:
a plurality of discrete sheets combined into an ingestible unit, wherein each discrete sheet has a composition configured for one or more predetermined functions.

2.-5. (canceled)

6. The ingestible unit of claim 1, wherein the plurality of discrete sheets are substantially identical.

7. The ingestible unit of claim 1, wherein one or more of the plurality of discrete sheets are at least one of water-dissolvable or water-stable.

8. (canceled)

9. The ingestible unit of claim 1, wherein one or more discrete sheets are formed from at least one of a polymer, polysaccharide, a film-forming protein, or an inorganic compound.

10.-14. (canceled)

15. The ingestible unit of claim 1, wherein one or more of the plurality of discrete sheets are at least one of films, laminas, or wafers.

16.-17. (canceled)

18. The ingestible unit of claim 1, wherein the plurality of discrete sheets is laminated together or contained within a coating.

19.-21. (canceled)

22. The ingestible unit of claim 1, wherein the ingestible unit is configured as at least one of a tablet, a sprinkle, a caplet, a placebo, or capsule.

23. The ingestible unit of claim 1, wherein at least one discrete sheet includes a biologically active agent.

24. (canceled)

25. The ingestible unit of claim 1, wherein the ingestible unit is configured to be at least one of orally-administrable or administered as a suppository.

26.-29. (canceled)

30. The ingestible unit of claim 1, the ingestible unit comprising a laminate body comprising a top discrete sheet, a bottom discrete sheet, and one or more discrete sheets therebetween.

31. The ingestible unit of claim 1, the ingestible unit comprising a laminate body comprising one or more of the following:
a sheet adhesive agent adhering adjacent discrete sheets together;
adjacent sheets of the plurality of discrete sheets include polymers that adhere together and adhere the adjacent discrete sheets together; or
adjacent discrete sheets of the plurality of discrete sheets include sheet adhesive agents that adhere the adjacent discrete sheets together.

32.-42. (canceled)

43. A method of making an ingestible unit, the method comprising:
obtaining at least one discrete sheet;
identifying a predetermined function for the at least one discrete sheet;
preparing the at least one discrete sheet to contain the predetermined function; and
combining a plurality of the discrete sheets into the ingestible unit.

44. (canceled)

45. The method of claim 43, further comprising encasing the plurality of discrete sheets in an ingestible container to form the ingestible unit.

46.-47. (canceled)

48. The method of claim 43, further comprising preparing one or more of the discrete sheets to be films.

49. The method of claim 43, further comprising:
stacking the plurality of discrete sheets to form a stacked plurality of discrete sheets;
adhering the plurality of discrete sheets together;
pressing the plurality of discrete sheets together; and
laminating the plurality of discrete sheets into a laminated body.

50.-53. (canceled)

54. The method of claim 43, further comprising:
forming each discrete sheet by at least one of: casting; spin coating; extrusion and calendering; roll-pressing; microfabrication; molding; jet-printing; drop-on demand; or dot printing.

55. (canceled)

56. The method of claim 43, further comprising:
determining a dose of a biologically active agent to be included in at least one of the plurality of discrete sheets of the ingestible unit;
determining an amount of the biologically active agent in a discrete sheet;
determining a number of discrete sheets having the biologically active agent to combine to obtain the determined dose; and
combining the number of discrete sheets having the biologically active agent in the ingestible unit.

57. (canceled)

58. The method of claim 43, further comprising:
preparing a plurality of particles having a biologically active agent;
suspending the plurality of particles in a composition;
forming the composition into a discrete sheet; and
including the discrete sheet in the ingestible unit.

59. The method of claim 43, comprising:
combining two different discrete sheets having two different biologically active agents into the ingestible unit.

60.-69. (canceled)

70. A method of providing an ingestible unit to a subject, the method comprising:
providing an ingestible unit having a plurality of discrete sheets combined into the ingestible unit, wherein each discrete sheet has a composition configured for one or more predetermined functions; and
administering the ingestible unit to a subject.

71.-73. (canceled)
